(19) United States
(12) Patent Application Publication
(10) Pub. No.: US 2026/0054052 A1
(43) Pub. Date: Feb. 26, 2026

(54) DIRECT CARDIAC COMPRESSION DEVICE WITH PERICARDIAL ELECTRODES

(71) Applicants: CorInnova, Inc., Houston, TX (US); THE TEXAS A&M UNIVERSITY SYSTEM, College Station, TX (US)

(72) Inventors: Megan Elizabeth Haynes, League City, TX (US); Erica Christine Perez, West Hartford, CT (US); Michael T. McDowall, Sugar Land, TX (US); Graham Michael Weeks, Rosharon, TX (US); Christina M. Bolch, Houston, TX (US); Boris Leschinsky, Mahwah, NJ (US); John C. Criscione, College Station, TX (US)

(73) Assignees: CorInnova, Inc., Houston, TX (US); THE TEXAS A&M UNIVERSITY SYSTEM, College Station, TX (US)

(21) Appl. No.: 18/815,474

(22) Filed: Aug. 26, 2024

(51) Int. Cl. A61M 60/515; A61M 60/191; A61M 60/289; A61M 60/468

(52) U.S. Cl. CPC A61M 60/515; A61M 60/191; A61M 60/289; A61M 60/468

(57) ABSTRACT

A direct cardiac compression device includes a flexible outer layer configured to fit about at least a portion of a living heart and contains at least a first pericardial electrode facing outward therefrom. The pericardial electrode is configured to detect an electrocardiogram of the living heart. The device further includes at least one inflatable active chamber inside the flexible outer layer facing the living heart, and a port operably connected with the at least one inflatable active chamber and configured to facilitate inflation and deflation of the at least one active chamber by an actuator to cause periodic direct compression of the living heart. The actuator may be controlled to inflate and deflate the at least one active chamber using the electrocardiogram signal collected from the first pericardial electrode.

### TECHNICAL FIELD OF THE INVENTION

The present invention relates in general to the field of direct cardiac compression devices and more specifically to direct cardiac compression devices having pericardial electrodes.

### CROSS-REFERENCE TO RELATED APPLICATIONS

None.

### STATEMENT OF FEDERALLY FUNDED RESEARCH

None.

### INCORPORATION-BY-REFERENCE OF MATERIALS FILED ON COMPACT DISC

None.

### BACKGROUND OF THE INVENTION

Without limiting the scope of the invention, its background is described in connection with heart assist devices. More particularly, the invention describes a direct cardiac compression device equipped with at least one pericardial electrode configured for the detection of the electrocardiogram of the heart.

Heart failure (HF) is a complex clinical syndrome where the heart's ability to pump blood is insufficient to meet the body's metabolic needs. This condition has become a significant public health challenge in the United States, with its prevalence showing a marked increase over recent decades. As of the latest data from the American Heart Association, approximately 6.2 million American adults live with heart failure, and this number is expected to rise due to several contributing factors.

One of the primary drivers of the increasing prevalence of heart failure is the aging population. The incidence of heart failure increases with age, particularly among individuals over 65, who are at higher risk due to age-related changes in cardiovascular physiology, including reduced myocardial elasticity and increased arterial stiffness. As the proportion of older adults in the U.S. population grows, so does the prevalence of heart failure.

Advancements in medical technology and improved management of acute cardiovascular conditions, such as myocardial infarctions and coronary artery disease, have also contributed to the rising number of heart failure cases. While these advancements have significantly decreased mortality rates from acute cardiac events, they have concurrently increased the number of individuals surviving these events, who are at a heightened risk of developing chronic heart failure over time.

Comorbid conditions such as hypertension, diabetes mellitus, and obesity are also major risk factors for heart failure, and their increasing prevalence further exacerbates the HF burden. Hypertension, or high blood pressure, leads to an increased workload on the heart, eventually causing hypertrophy and heart failure. Diabetes mellitus contributes to HF through various mechanisms, including diabetic cardiomyopathy and an increased risk of coronary artery disease. Obesity is linked to heart failure due to its association with other risk factors like hypertension, diabetes, and metabolic syndrome.

The economic impact of heart failure is substantial, with direct and indirect costs estimated to be in the billions of dollars annually. These costs encompass hospitalizations, outpatient visits, medications, and lost productivity. The increasing prevalence of heart failure is projected to result in a dramatic rise in healthcare expenditures over the next few decades, necessitating more efficient and cost-effective management strategies.

Effective management and prevention of heart failure involve a multifaceted approach. Lifestyle modifications, such as dietary changes, regular physical activity, and smoking cessation, are foundational strategies for reducing risk factors. Pharmacological interventions, including ACE inhibitors, beta-blockers, diuretics, and newer agents like angiotensin receptor-neprilysin inhibitors (ARNIs), are essential in managing symptoms and improving outcomes. Additionally, the implementation of innovative healthcare delivery models, such as heart failure clinics and telemedicine, can enhance patient care and reduce hospital readmissions.

The management of advanced heart failure (HF) increasingly involves the utilization of mechanical circulatory support (MCS) devices, which may be broadly categorized into short-term and long-term assist devices. These devices play a critical role in stabilizing and supporting patients with severe cardiac dysfunction. Short-term heart assist devices, such as intra-aortic balloon pumps (IABP), extracorporeal membrane oxygenation (ECMO), and percutaneous ventricular assist devices (pVADs), are primarily employed in acute settings to provide temporary hemodynamic support. IABPs enhance coronary perfusion and reduce cardiac workload by inflating and deflating a balloon in the aorta synchronized with the cardiac cycle. ECMO provides both cardiac and respiratory support by oxygenating blood outside the body and returning it to the patient, thus allowing the heart and lungs to rest and recover. pVADs, such as the Impella device, are inserted percutaneously and directly unload the left ventricle, improving cardiac output and systemic perfusion in acute cardiogenic shock or high-risk percutaneous coronary interventions.

Long-term heart assist devices, specifically left ventricular assist devices (LVADs), are increasingly utilized for patients with end-stage heart failure as a bridge to transplantation or as destination therapy for those ineligible for a heart transplant. LVADs are implanted surgically and consist of a pump that assists the left ventricle in circulating blood throughout the body, thereby reducing symptoms, improving quality of life, and extending survival. Modern LVADs, such as the HeartMate 3, incorporate features like magnetically levitated rotors to minimize wear and thrombogenesis, thereby enhancing device durability and patient outcomes. The use of LVADs requires careful patient selection, comprehensive management of anticoagulation to prevent thromboembolic complications, and vigilant monitoring for device-related issues such as infection, bleeding, and right ventricular failure.

The integration of these heart assist devices into clinical practice has revolutionized the management of advanced heart failure, offering life-saving support and bridging patients to recovery, transplantation, or long-term therapy. Ongoing advancements in device technology, improved patient management protocols, and interdisciplinary care teams are essential in optimizing the outcomes and quality of life for patients with severe heart failure.

MCS devices, while revolutionary in the management of advanced heart failure, have several notable limitations that pose significant challenges. One major limitation is the need for extensive implantation and, in some cases, explantation surgeries. These procedures are highly invasive, requiring significant surgical expertise and resources. The implantation of devices like left ventricular assist devices (LVADs) involves opening the chest cavity, cannulating major blood vessels, removing a portion of heart muscle or great vessels, and ensuring precise placement and secure attachment of the device components. This extensive surgical intervention carries inherent risks, including surgical site infections, bleeding, embolism, and prolonged recovery times, which can be particularly taxing for patients who are already in a fragile state due to their advanced heart failure or advanced age.

Additionally, the interaction of MCS devices with blood can lead to several complications. The contact between blood and the artificial surfaces of these devices can trigger a cascade of adverse hematologic events. One significant issue is thrombogenesis, where blood clots can form on or within the device. These clots pose a serious risk of embolic events, such as strokes or systemic embolisms, necessitating the use of anticoagulation therapy to mitigate this risk. However, anticoagulation therapy itself introduces a risk of bleeding, complicating the management of these patients.

Hemolysis, the destruction of red blood cells, is another complication associated with MCS devices. This occurs due to the mechanical stress exerted on blood cells as they pass through the device, leading to anemia and further complicating the patient's condition. Furthermore, the continuous flow provided by many MCS devices can alter normal hemodynamic parameters, potentially leading to issues such as gastrointestinal bleeding and aortic insufficiency.

Direct cardiac compression devices (DCCD) present a promising less-invasive and non-blood-contacting alternative to traditional heart assist blood pumps. These devices are designed to mechanically support the heart by externally compressing it, thus augmenting its pumping and filling action without directly contacting the blood, thereby avoiding many of the complications associated with blood-contacting devices.

One of the primary advantages of direct cardiac compression devices is their less invasive nature. Unlike left ventricular assist devices (LVADs) that require major open-heart surgery for implantation, direct cardiac compression devices can often be implanted through less invasive procedures without cardiopulmonary bypass, potentially even via minimally invasive surgical techniques. This reduces the surgical risk and recovery time for patients, making these devices a viable option for those who may not be suitable candidates for more invasive procedures.

Direct cardiac compression devices work by applying mechanical pressure to the heart in a synchronized manner with the cardiac cycle. This external compression helps enhance the natural contractile function of the myocardium, effectively increasing cardiac output and improving systemic perfusion. Because these devices do not come into direct contact with blood, they eliminate the risks of thrombogenesis, hemolysis, and other blood-contact-related complications. This significantly reduces the need for long-term anticoagulation therapy, thereby lowering the risk of bleeding complications that are commonly associated with blood-pumping devices.

Furthermore, the external position of these devices with regard to the heart and vasculature means there is a lower risk of device-related infections. Since the device components are not implanted within the bloodstream, the risk of bacterial colonization and subsequent infections is minimized, along with reduced systemic inflammation. This can result in a more favorable long-term outcome for patients, with fewer complications and hospital readmissions.

Examples of DCCD devices are described in the following US Patents and Patent Applications incorporated herein by reference: U.S. Pat. No. 10,123,875, US20200360583, US20170080137, and US20230355956. A general DCCD is shown in FIG. 1. It may include a flexible inflatable body 20 that may be positioned to fit and surround at least a portion of the heart 10, such as at least a portion of area 14 of the heart containing at least a part of the left ventricle and at least a part of the right ventricle. It may also fit about an apex area 12. The device may be implanted and positioned between the epicardial surface of the heart 10 and the pericardium 13. An optional hub 26 may be provided to serve as a base for all components of the DCCD. A first electrocardiogram-sensing electrode 22 may be positioned on the most inner surface of the flexible inflatable body 20 to face the epicardial surface of the heart 11. An electrode conductor 24 may be attached to the first electrode 22 and may be configured to transmit the electrocardiogram signal from the first electrode 22 to the controller of the direct cardiac compression device. The controller may be configured to operate an actuator and cause periodic inflations and deflations of the flexible inflatable body 20 causing compression of the heart 10 in the area 14. Such compressions may be synchronized with the natural rhythm of systolic contractions of the living heart. This controller and the actuator may be external to the body of the patient, such as a bedside or a portable controller. In other versions, the controller and the actuator may be incorporated with the DCCD making the entire system fully implantable.

Despite their non-blood contact nature and other advantages, direct cardiac compression devices are not without their limitations. Ensuring precise synchronization with the cardiac cycle and achieving effective compression without causing damage to the myocardium or surrounding tissues can be challenging. In particular, reliable acquisition of the electrocardiogram signal from the epicardial surface of the heart is not an easy task given the wide variety of heart sizes and shapes that these devices are designed to fit about. Prior art devices describe the use of one or more epicardial electrodes facing inward from the device and configured to touch the epicardial surface of the living heart. Epicardial surface of the heart may be sensitive to the presence of such electrodes. Epicardial electrodes have a potential to irritate the heart surface, which may result in a disruption of normal myocyte polarization/conduction. Furthermore, since the epicardial heart surface may be irregular, as seen in area 15, there is a risk of incomplete or inconsistent contact between the generally flat epicardial electrode and the irregular heart surface. In addition, there is a risk of an adhesion between the electrode and the heart with normal tissue response to the implantable, or abrasion to the epicardium caused by the electrodes, which may present a problem when the DCCD is removed or potentially disrupt normal myocardial conduction.

The need exists therefore for a direct cardiac compression device with improved ability to reliably acquire noise-free electrocardiogram signals without interruptions and erroneous readings and minimal heart (epicardial) tissue-device interface reactions prior to DCCD removal.

### BRIEF SUMMARY OF THE INVENTION

The present invention provides a direct cardiac compression device comprising: a flexible outer layer configured to fit about at least a portion of a living heart, wherein the flexible outer layer comprises a first pericardial electrode facing outward therefrom, the first pericardial electrode is configured to detect an electrocardiogram of the living heart, at least one inflatable active chamber inside the flexible outer layer and facing the living heart, a port operably connected with the at least one inflatable active chamber and configured to facilitate inflation and deflation of the at least one inflatable active chamber by an actuator to cause periodic direct compression of the living heart, wherein the actuator is controlled to inflate and deflate the at least one inflatable active chamber using the electrocardiogram signal collected from the first pericardial electrode.

In another embodiment the present invention provides a direct cardiac compression device comprising: a flexible outer layer comprises an outer surface and an inner surface, wherein the inner surface is configured to fit about at least a portion of a living heart; a first pericardial electrode in contact with the outer surface and facing away from the inner surface, wherein the first pericardial electrode is configured to detect an electrocardiogram of the living heart; at least one inflatable active chamber inside the flexible outer layer and facing the living heart, a port operably connected with the at least one inflatable active chamber and configured to facilitate inflation and deflation of the at least one inflatable active chamber by an actuator to cause periodic direct compression of the living heart, wherein the actuator is controlled to inflate and deflate the at least one active chamber using the electrocardiogram signal collected from the first pericardial electrode.

The present invention provides a direct cardiac compression device configured to fit about at least a portion of a living heart, the direct cardiac compression device comprising: one or more passive chambers facing the living heart and shaped to taper toward an apex of the living heart, a plurality of inflatable active chambers externally adjacent to the one or more passive chambers, wherein inflatable active chambers are shaped to taper toward the apex of the living heart and at least partially surround the one or more passive chambers, wherein when inflated, the plurality of inflatable active chambers are configured to cause direct cardiac compression of the living heart, a self-expandable wireframe positioned to surround and configured to support the plurality of inflatable active chambers, wherein the self-expandable wireframe extends from a hub, a first port operably connected to the plurality of inflatable active chambers to independently inflate and deflate thereof, a second port operably connected to the at least one or more passive chambers to inject or adjust a volume of fluid therein, and a flexible outer layer configured to surround the one or more passive chambers, the plurality of active chambers, and the self-expandable frame, wherein the outer layer comprises a first pericardial electrode facing outward therefrom, the first pericardial electrode is configured to detect an electrocardiogram of the living heart.

### BRIEF DESCRIPTION OF THE SEVERAL VIEWS OF THE DRAWINGS

Subject matter is particularly pointed out and distinctly claimed in the concluding portion of the specification. The foregoing and other features of the present disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1 is a general schematic view of a direct cardiac compression of the prior art,

FIG. 2 is a general schematic view of a direct cardiac compression of the present invention,

FIG. 3 is a schematic cross-section of a first embodiment of the direct cardiac compression device,

FIG. 4 is a schematic cross-section of a second embodiment of the direct cardiac compression device,

FIG. 5 is a schematic cross-section of a third embodiment of the direct cardiac compression device,

FIG. 6 is a general side view showing the positions of pericardiac electrodes of the DCCD, facing outward,

FIG. 7 is a close-up perspective view of the electrode of the present invention, FIG. 8 is a top internal view of the same,

FIG. 9 is an external view of electrode subassembly,

FIG. 10 is a partial cross-sectional view of the electrode protrusions enclosed in a flexible film,

FIG. 11 is a partial cross-sectional view of the electrode and electrode conductor embedded in a flexible film,

FIG. 12 is a close-up of the electrode embedded in the flexible outer layer of the DCCD,

FIG. 13 is an example of a typical electrocardiogram obtained from the skin of a subject,

FIG. 14 is an example of a typical electrocardiogram obtained from an epicardial surface of a subject, and

FIG. 15 is an example of a typical electrocardiogram obtained from the inner pericardial surface of a subject.

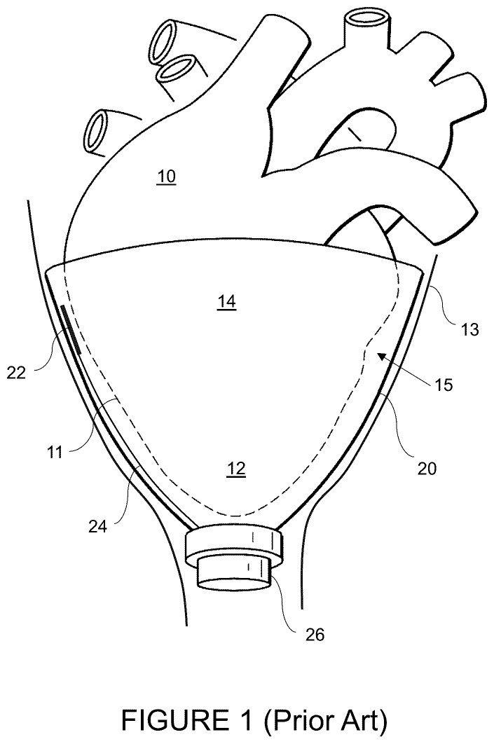

### DETAILED DESCRIPTION OF THE INVENTION

The following description sets forth various examples along with specific details to provide a thorough understanding of the claimed subject matter. It will be understood by those skilled in the art, however, that claimed subject matter may be practiced without one or more of the specific details disclosed herein. Further, in some circumstances, well-known methods, procedures, systems, components and/or circuits have not been described in detail in order to avoid unnecessarily obscuring the claimed subject matter. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and make part of this disclosure.

Accordingly, it is an object of the present invention to overcome these and other drawbacks of the prior art by providing a novel direct cardiac compression device with an improved ability to acquire electrocardiogram signals needed for consistent synchronization of the compressive actions of the device and systolic contractions of the living heart.

It is another object of the present invention to provide a novel direct cardiac compression device with sensing electrodes configured for reliable acquisition of the electrocardiogram signals in a manner that does not depend on consistent and full contact with the epicardial surface of the living heart.

It is a further object of the present invention to provide a novel direct cardiac compression device that may be implanted to fit about the heart in any rotational orientation while supporting consistent detection and acquisition of the electrocardiogram of the living heart.

It is yet a further object of the present invention to provide a novel direct cardiac compression device that minimizes tissue adhesion and improves the ability to remove the device after use without any heart damage.

It is a further object of the present invention to reduce a risk of epicardial abrasion by the heart assist device, as well as disruptions in myocardial conduction that may result from such abrasion.

The direct cardiac compression device of the invention may include a flexible outer layer configured to fit about at least a portion of a living heart. The flexible outer layer may include a first pericardial electrode facing outward therefrom and configured to detect an electrocardiogram of the living heart. The device may further include at least one inflatable active chamber inside the flexible outer layer and facing the living heart. Furthermore, the device may feature a port operably connected with the at least one inflatable active chamber and configured to facilitate inflation and deflation of the at least one active chamber by an actuator to cause periodic direct compression of the living heart. The actuator may be controlled to inflate and deflate the at least one active chamber using the electrocardiogram signal collected from the first pericardial electrode.

In other embodiments, a direct cardiac compression device configured to fit about at least a portion of a living heart, may include one or more passive chambers facing the living heart and optionally shaped to taper toward an apex of the living heart. It may further include one or a plurality of inflatable active chambers connected to the one or more passive chambers. The inflatable active chambers may be shaped to taper toward the apex of the living heart and at least partially surround the one or more passive chambers. When inflated, the one or more inflatable active chambers may be configured to cause direct cardiac compression of the living heart.

The DCCD may further optionally include a self-expandable frame, which may be positioned to be in contact with to support at least some or with each of the plurality of inflatable active chambers. The frame may extend from an optional hub of the device. The hub, in turn, may include at least a first port operably connected to the one or more inflatable active chambers to independently inflate and deflate thereof. The hub may in some cases also feature a second port operably connected to the at least one or more passive chambers and configured to inject or adjust a volume of fluid therein. Additional ports may be also provided to individually inflate different chambers or groups of chambers of the device if desired, as the invention is not limited in this regard. Other embodiments may not contain the hub as various lines, tubes, and electrical conductors may be routed individually or in suitable bundles that may not traverse the hub.

The DCCD may define a flexible outer layer configured to surround the one or more passive chambers, the plurality of active chambers, and the self-expandable frame. The outer layer comprises a first pericardial electrode or a plurality of pericardial electrodes facing outward therefrom. The first pericardial electrode or the plurality of pericardial electrodes may be configured to detect an electrocardiogram of the living heart.

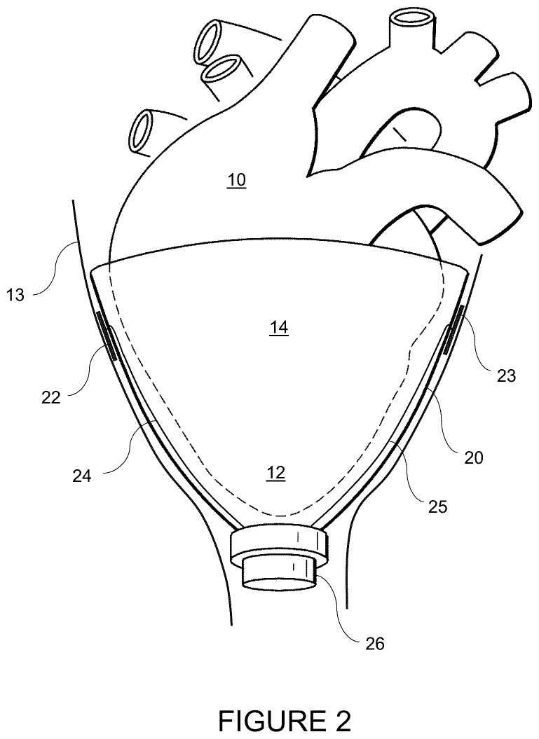

FIG. 2 shows a general schematic view of the present invention. To improve the reliability and consistency of the detection and acquisition of the electrocardiogram and to limit abrasive or irritating interface with the epicardium, at least one pericardial electrode 22 of the DCCD device is now placed on the flexible outer layer of the device to face the inner surface of the pericardium.

Pericardial tissue, which envelops the heart, exhibits several distinct physical properties essential for its physiological function. It is primarily composed of two layers: the fibrous pericardium and the serous pericardium. The fibrous pericardium is a tough, inelastic connective tissue layer that provides structural integrity and anchors the heart within the thoracic cavity, preventing overdistension during increased blood volume. This layer is composed of dense collagen fibers, which confer high tensile strength, allowing it to withstand substantial mechanical stress. The serous pericardium, lying beneath the fibrous layer, is divided into two sublayers: the parietal layer, which lines the inner surface of the fibrous pericardium, and the visceral layer, which covers the heart's surface. These layers are composed of a thin, mesothelial cell lining that secretes a lubricating serous fluid into the pericardial cavity.

The pericardial tissue exhibits elasticity and compliance, crucial for accommodating the dynamic changes in heart size and shape during contraction and relaxation. The tissue's elasticity is attributed to the presence of elastin fibers interspersed within the collagen matrix. Moreover, the pericardial layers are richly innervated and vascularized, ensuring adequate nutrient delivery and waste removal, and they contain lymphatic vessels that aid in fluid regulation.

The invention takes advantage of the great flexibility and yet limited elasticity of the pericardial tissue. The at least one pericardial electrode 22 facing the inner surface of the pericardium, namely the serous pericardium layer, may be positioned to reliably stay in contact with the inner pericardium surface so as to consistently provide the required function of sensing the electrocardiogram of the living heart.

While the pericardium is present in most patients requiring the use of direct cardiac compression devices, there may be some cases when the pericardium is removed, partially excised or is absent altogether-such as in a case of a patient with a history of prior heart surgery. In these cases, the at least first pericardial electrode 22 facing away from the heart assist device will still be in contact with the tissues surrounding the heart to support sensing the electrical activity of the heart. In that sense, the term “pericardial electrode” is used in this description to describe any electrode facing outward from the device and the living heart inside thereof.

In one embodiment the at least first pericardial electrode 22 may be made to be flush with the flexible outer layer 20 to avoid creating any abrasion of the pericardial surface and to minimize the opportunity for tissue adhesion, for example, during insertion, implant duration, and removal of the device. Note that while illustrations in FIGS. 2-4 show pericardial electrodes as adjacent to or even as separated and slightly spaced apart from the flexible outer layer, this is done only for the purposes of the illustration clarity and does not reflect the actual design intent of the devices of the present invention.

Furthermore, the first pericardial electrode 22 may be made to have a smooth electroconductive surface facing the pericardium, as explained in greater detail below. The size of the electrically sensitive surface of the first pericardial electrode 22 may be selected to be sufficient for detecting the electrocardiogram of the living heart, as known in the art of ECG-sensing electrodes.

Electrocardiogram detected by the first pericardial electrode may be transmitted using the first electrode conductor 24, which may be routed along the periphery of the direct cardiac compression device either inside or outside thereof, as described in greater detail below. The electrode conductor 24 may be made using a single- or in some embodiments a multi-strand wire made from a highly conductive material, such as silver or like metal. The electrode conductor 24 may be coated or enclosed within an insulating jacket.

Additional pericardial electrodes may be provided and located on the flexible outer layer 20 of the DCCD device facing externally toward the pericardial tissue. FIG. 2 shows an example of a second pericardial electrode 23 located on the outer flexible layer 20 and facing away from the living heart 10. The second pericardial electrode 23 may have a dedicated second electrode conductor 25 configured to transmit the electrical signal from the second pericardial electrode 23 to the controller of the device. In some embodiments, two pericardial electrodes may be located opposite each other as seen in FIG. 2. This may be advantageous to provide detection of electrical activity at various locations around the living heart.

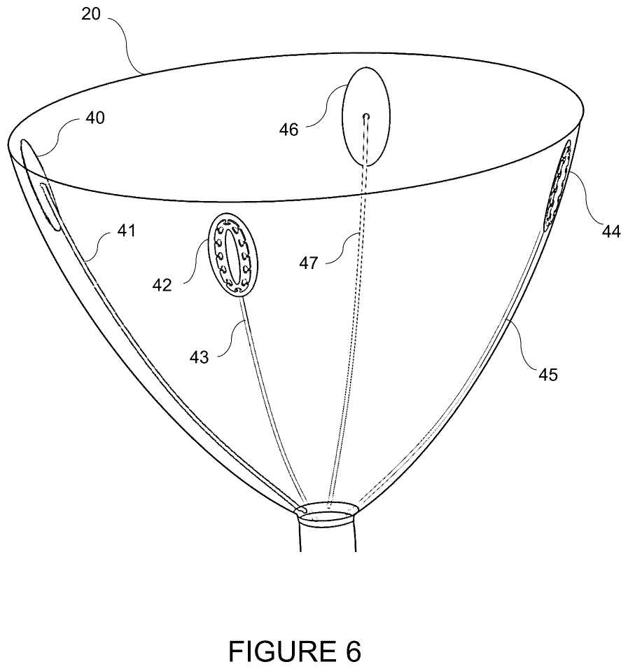

Further additional pericardial electrodes may be provided as seen, for example, in FIG. 6. In various embodiments, a total number of pericardial electrodes may be at least one, at least two, at least three, at least four, at least five, at least six, or even more as may be needed for more complete and reliable detection of the electrogram of the entire heart or, in some cases, collecting individual electrograms of certain portions of the heart. Examples of dedicated electrocardiogram sensing for individual portions of the heart may include sensing of the left bundle branch, right bundle branch, and other individual electrical conductive pathways along the living heart. In further embodiments, the presence of more than one pericardial electrode may be used as a redundancy measure to increase the reliability of operation and provide multiple ways to detect the electrical activity of the heart, as the invention is not limited in this regard.

Multiple electrodes may be evenly spread around the periphery and/or along the central axis of the heart assist device of the present invention so that insertion of the device in any radial orientation would not cause any preferential detection of the electrocardiogram on one side of the living heart or another. Furthermore, in some embodiments, multiple pericardial electrodes may be positioned in accordance with a 3-lead configuration interface designed for the triangle between RA, LA, LL. In other embodiments, multiple pericardial electrodes may be positioned around and along the device in accordance with a scheme configured for interfacing with 5-lead ECG sampling of RA, RL, LA, LL, V1. Other positions of pericardial electrodes may also be provided, as the invention is not limited in this regard.

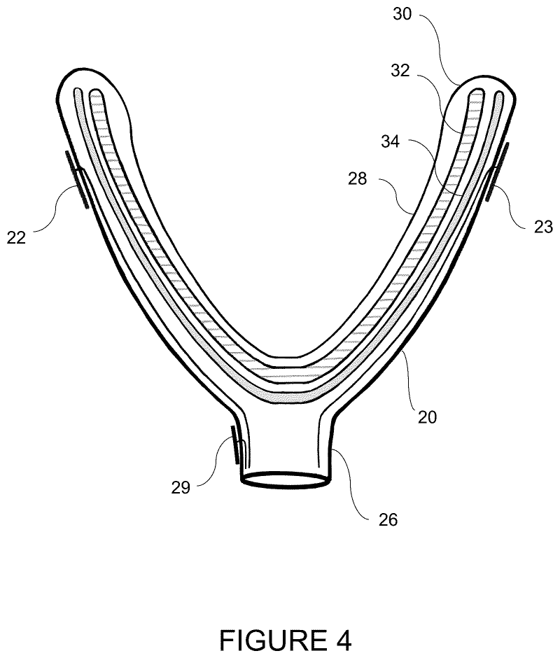

In addition, a base or ground electrode may be located at or adjacent to the hub of the device so that electrocardiogram sensing may be done between that ground electrode and at least one or more pericardial electrodes facing outward from the heart. One example of a ground electrode 29 is seen in FIG. 4. If the wireframe is at least partially exposed to outer tissues such as pericardium, the wireframe or components thereof may also be used as the ground electrode. In other embodiments, the ground electrode may also be attached directly to the tissue/chest cavity and not associated with the invention's heart assist device.

The at least first and/or additional pericardial electrodes may also serve as convenient radiopaque markers to identify the position of the heart assist device on X-Ray and fluoroscopy images.

In further embodiments, at least one epicardial electrode may be present on the internal surface of the device facing the epicardial surface of the heart, if there is a need to detect epicardial electrical activity or pace the heart, as the invention is not limited in this regard.

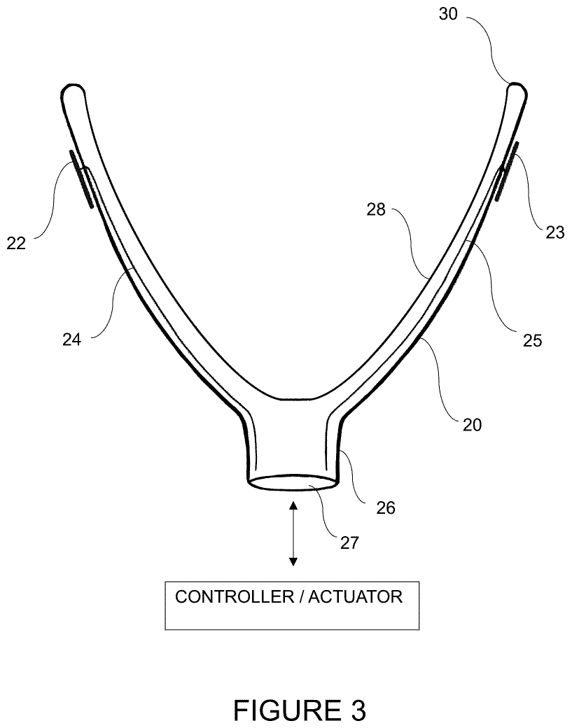

Direct cardiac compression devices may be provided in various configurations. In all of these configurations, the at least first pericardial electrode 22 as well as additional pericardial electrodes may be positioned on the flexible outer layer so as to face outward from the living heart and toward the pericardium or other external tissue. FIG. 3 shows the first embodiment of the DCCD comprising an inflatable compartment 30 defined by the flexible inner layer 28 and flexible outer layer 20. Both the inner layer 28 and the outer layer 30 may be made from a flexible material such as a thin polymer film in order to compress the entire device prior to its implantation to facilitate minimally invasive insertion as well as removal after the therapy is complete. If the device is intended to be inserted and removed using an open chest surgery, it is also possible to have the outer layer 20 to be less flexible, as the invention is not limited in this regard.

While in some embodiments, the inflatable compartment 30 may feature a single inflatable active chamber, in other embodiments, it may feature a plurality of inflatable active chambers, such as at least two, at least three, at least four, at least five, at least six, at least seven, at least eight, or even more active chambers, all shaped to surround the living heart. Multiple inflatable active chambers may be positioned adjacent to each other and, in some cases, may partially overlap each other. The one or more inflatable active chambers, when inflated, are configured to apply external compression to the living heart and to assist the heart to eject blood therefrom.

Various flexible biocompatible thin film polymer materials may be used to make the at least one active chamber of the inflatable compartment 30. Examples of such materials include various polyurethanes, polyethylenes, polyesters, silicones, as well as blends and combinations thereof, as the invention is not limited in this regard. At least a portion of or the entire thin polymer film of various components of the DCCD may contain fiber reinforcement. Fiber reinforcement may be embedded within, attached to one side of the thin film, or positioned adjacent thereto. Fiber reinforcement may be made as a thin mesh of small-diameter strands and may be configured to prevent undesired expansion of the thin polymer film. One example of such undesired expansion is an outward expansion of the inflatable active chambers of the device away from the heart, which may reduce heart assist efficacy during the therapy. Examples of such reinforcement structures are described in greater detail in other patent filings of the assignee of the present invention.

The inflatable compartment 30 may be shaped to converge at a port 27 located, for example, at the hub 26 positioned near the apex of the heart, and configured to be operably connected with the at least one active chamber of the inflatable compartment 30. The port 27 may be used as a conduit to facilitate inflation and deflation of the active chambers of the DCCD by the actuator as directed by the controller operating thereof. The actuator, in turn, may be configured to inject and withdraw air or another operating fluid, for example, saline or silicone oil, in order to inflate the active chambers and cause periodic direct compressions of the heart.

The hub 26 may also be used to route the electrode conductors from the respective pericardial electrodes to the controller. In this case, the controller may be configured to cause the actuator to inflate and deflate the active chambers of the direct cardiac compression device using the electrocardiogram signal or signals collected from the first or more pericardial electrode.

FIG. 4 shows an example of a second embodiment of the direct cardiac compression device having at least one pericardial electrode 22 of the present invention. This device comprises at least one inflatable active chamber or a plurality of inflatable active chambers 34 and at least one optional passive chamber or a plurality of passive chambers 32, all enclosed in a flexible containment layer 30. The one or more active chamber 34 may be inflated and deflated through at least one or more ports (not shown) configured to provide fluid communication with the actuator. The passive chambers 32 may be located inside the active chambers 34 and may be partially inflated with saline or another incompressible fluid during the implantation of the device around the heart. Such inflation may be done via a dedicated passive chambers port (not shown). The purpose of the passive chambers 32 is to fill and occupy the empty space or voids between the active chambers 34 and the living heart. Such empty space may be formed in case of some irregularities of the heart surface or in case of a small mismatch between the size of the DCCD and the size of the heart. Inclusion of the passive chambers may not diminish the inflation and deflation of the active chambers but may rather improve the direct transmission of compressive forces from the active chambers to the epicardial surface of the heart.

One purpose of providing a containment layer 30 is to envelope all other components of the device as shown in FIG. 4 and to contain any fluid leaks that may develop during the operation of the device. A fluid withdrawal port may be provided to remove such fluid in case a small leak is detected. Another purpose of providing a containment layer 30 is to define the boundaries for the DCCD device around the heart. In some embodiments it is useful in case a replacement of the entire device or some of its components if needed, for example, in case of a device failure. Such replacement would be accomplished by slipping the device out from the space within the containment layer and inserting a new device into the same space. In further embodiments, while the device may be removed after the therapy is completed, the containment layer may be left in place, for example, if another round of heart assist therapy is needed in the future.

At least one pericardial electrode 22 may be positioned on the flexible outer layer 20 of the containment layer 30 as seen in FIG. 4. The corresponding electrode conductor 24 may be routed from the first pericardial electrode 22 toward the hub 26 and further to the controller (not shown), as described below in further detail. A second pericardial electrode 23 may also be provided, as seen in FIG. 4.

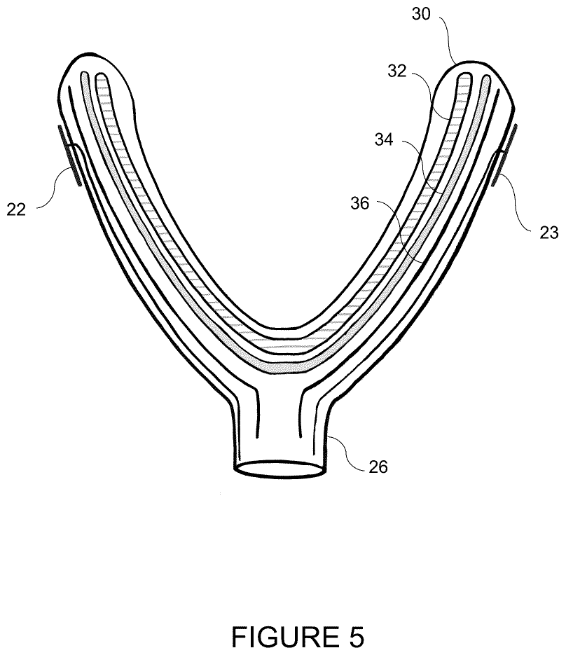

A third exemplary embodiment of a useful configuration of the direct cardiac compression device is seen in FIG. 5. This configuration has the same elements as the device seen in FIG. 4 with the addition of a wireframe 36. The wireframe 36 may be made from a plurality of self-expandable wires, such as NiTi wires, and may serve to facilitate minimally invasive insertion of the device. The wireframe 36 may also be useful in containing the outward expansion of the active chambers 34 and directing their inflation to be predominantly inward so as to compress the heart. In addition, the wireframe may be useful in holding the thin film components of the device in their intended positions throughout device operation so as to prevent or minimize their unintended migration away from their initial positions. Further aspects and examples of wireframe 36 are described in greater detail in other patent filings of the assignee of the present invention.

Other device configurations are also intended to be included in the scope of the present invention. One example of an alternative configuration is a device that may include a wireframe with optional passive chambers. This device may not include active chambers and therefore may be referred to as a “passive” assist device, which may be configured to assist the heart during diastole.

The details of electrode design and attachment to the flexible outer layer are now described with reference to FIGS. 6-12. FIG. 6 shows a generic direct cardiac compression device 20 with four pericardial electrodes 40, 42, 44, and 46 spaced about the periphery of the device 20. The four pericardial electrodes may be located respectively in a first quadrant, a second quadrant, a third quadrant, and a fourth quadrant thereof, thereby forming a plurality of redundant pericardial electrodes for collecting the electrogram of the living heart from a plurality of locations around the flexible outer layer 20. Corresponding electrode conductors 41, 43, 45, and 47 may be used to transmit electrocardiogram signals from the pericardial electrodes to the device controller. These conductors may be placed along the inner surface and/or the outer surface of the flexible outer layer and attached thereto in at least one location between the pericardial electrode and the hub, embedded within the flexible outer layer, or routed outside thereof. In some embodiments, the electrode conductors may be painted onto the flexible outer layer using an electroconductive paint or coating. In further embodiments, they may be routed inside one or more dedicated channels formed along the flexible outer layer, so as to prevent tissue adhesion or encapsulation within adjacent tissues, as the invention is not limited in this regard.

As mentioned above, the positions of the electrodes on device 20 may be evenly distributed around the periphery so as to provide for the ability to insert device 20 in any radial orientation around the heart during implantation. The pericardial electrodes may be positioned at various heights along the device starting from being adjacent to the hub and including being adjacent to the basal peripheral edge of the device. In some embodiments, the position of the peripheral electrodes may be selected to be on the midportion of the device, on the basal portion of the device, or closer to the basal edge and away from the hub so as to improve contact consistency with the pericardial tissue or target ECG sensing from different anatomical sensing locations.

To support the ability of the device to be compressed and delivered through a small opening of the delivery system via a minimally invasive approach, the pericardial electrodes may be configured to be sufficiently flexible to be foldable. On the other hand, they need to have sufficient contact area with the pericardial tissue to provide for a low resistance sensing of electrical activities of the living heart. One advantageous way to accomplish these design objectives is to provide the pericardial electrode in a shape having a greater length oriented in a direction from the hub to the base of the device (which is perpendicular to the radial circumference of the flexible outer layer), as compared to a shorter width oriented in a direction around and along the radial circumference of the device. This shape may allow for better electrode compression with fewer folds during device implantation while still providing a sufficiently large tissue contact area so as to reduce the electrical impedance of the electrode itself. In one example, illustrated in the drawings, the pericardial electrode may be shaped as an oval with the long axis oriented along the length of the device 20.

In some embodiments, the size of the oval may be selected to have a long axis from about 10 to about 20 millimeters and a short axis from about 4 to about 10 millimeters. In one embodiment, the oval is about 17 mm by 7 mm.

In some embodiments, the pericardial electrode may be made from a conductive metal foil, such as made from the following metals: gold, silver, platinum, platinum-iridium alloy, copper, steel, or another highly conductive and biologically inert metal or a metal alloy. In further embodiments, the electrode may have a substrate that may be coated with a conductive layer comprising any of these metals or alloys thereof. In one example, a platinum-iridium foil may have a thickness selected to be from about 0.01 to about 0.05 millimeters. Other metal foils may be provided in other thickness ranges selected from about 0.005 to about 0.5 millimeters to ensure the ability of the foil to fold during device delivery without damaging thereof while also supporting sufficient overall mechanical integrity of the electrode. The foil of the pericardial electrode may be selected to have a smooth outer surface facing the external tissue. This may be useful to minimize tissue abrasion and adhesion during the implantation and the use of the device.

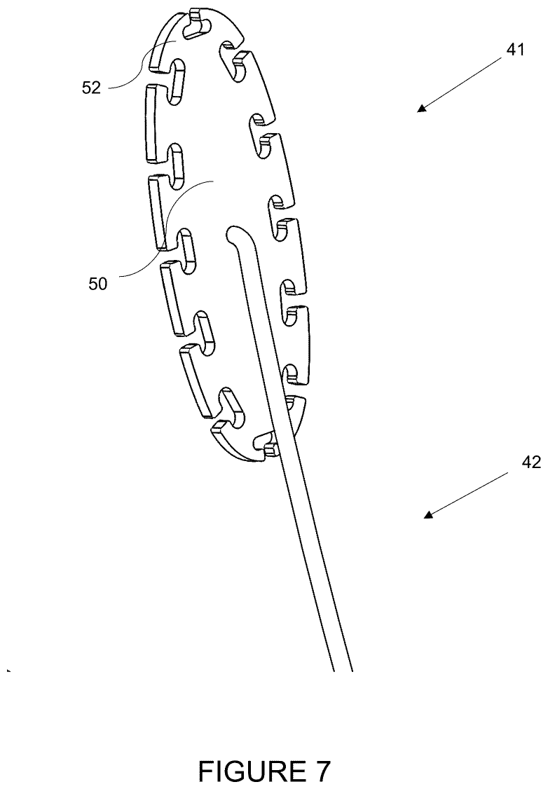

One exemplary design of the pericardial electrode 41 is seen in FIG. 7. The electrode may have the main conductive portion 50 surrounded by a plurality of radial protrusions 52. Radial protrusions 52 may be used to encapsulate the foil of the pericardial electrode 41 into the polymer thin film in a manner to (i) expose a sensing electroconductive surface to the pericardium, and (ii) avoid leakage into or out of the flexible outer layer of the device during its operation, and (iii) provide a smooth biocompatible surface exposed to tissues surrounding the heart.

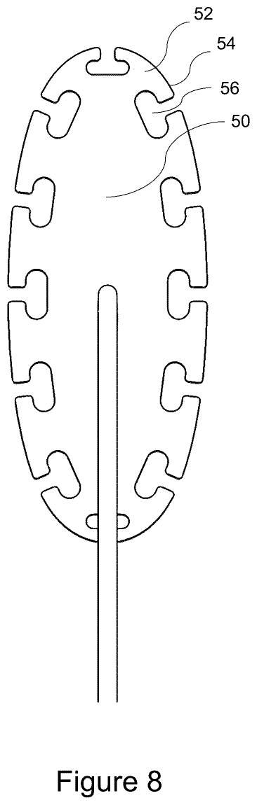

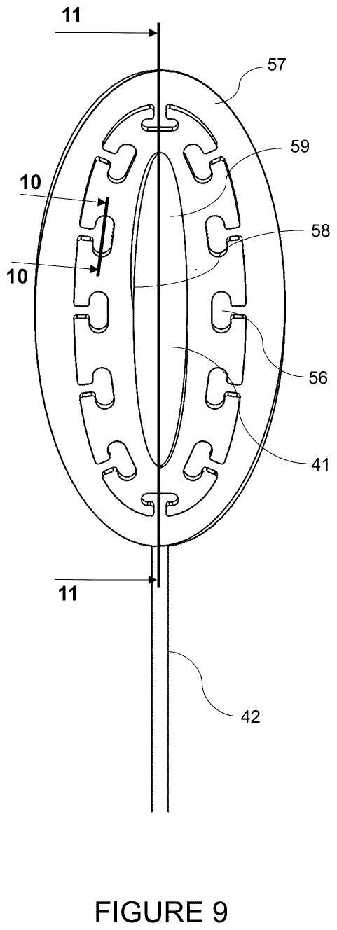

FIG. 8 illustrates the details of one advantageous design of radial protrusions 52 surrounding the main conductive portion 50 of the electrode 41. Each radial protrusion 52 may have a widened outer portion 54 attached to the main conductive portion 50 with a narrow arm to form a plurality of voids 56 around the periphery of the main conductive portion 50. To seal the foil electrode 41 between two layers of thin film 61 and 62 (see FIGS. 9, 10, and 11), the portions of the thin film layers 61 and 62 located over each other may be fused together using heat, pressure, ultrasound welding, or another film fusion and joining method. The thin film may be selected with a thickness ranging from about 0.0005 to 0.004 inches. As a result, radial protrusions 52 are trapped inside and secured between the fused portions 63 of the polymer film 61 and 62 and the entire foil electrode 41 is embedded into the film layers in a sealed manner.

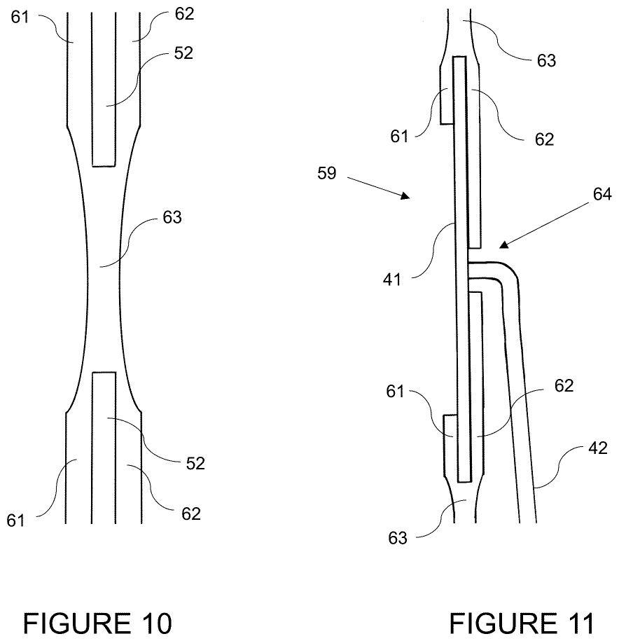

FIG. 10 shows a partial cross-sectional view of the two fused layers 61 and 62 along one of the voids 56, with FIG. 9 providing the cross-sectional cut 10 of the assembly depicted in FIG. 10. Adjacent sections of radial protrusions 52 are seen positioned within and between the thin film layers 61 and 62, with fused film 63 between the voids 56. A sensing window 59 may be formed in the external film layer 61 to expose the foil electrode 41 to the adjacent pericardial tissue, as demonstrated in FIG. 9 and FIG. 11. The sensing window 59 may be sized to be smaller than the main conductive portion 41 so as not to interfere with or overlap the plurality of voids 56. FIG. 11 demonstrates that the electrode conductor 42 may be soldered or otherwise mechanically and electrically attached to the opposite (internal) side of the foil electrode 41, such as through a small window 64 in the polymer film layer 62.

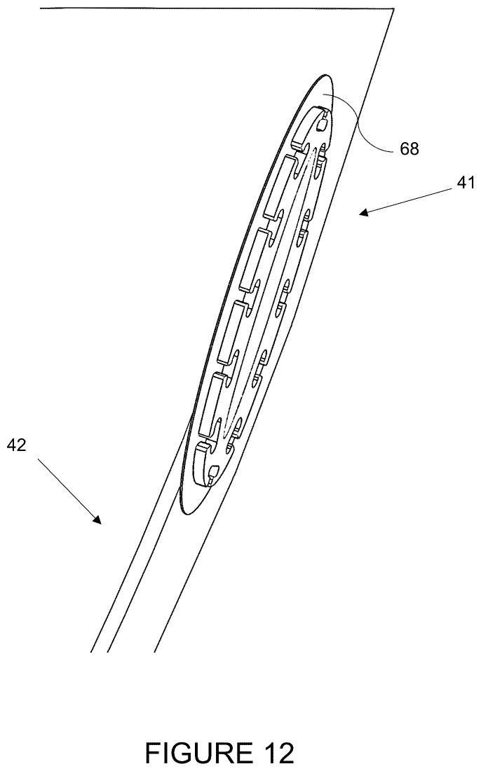

In some embodiments, the pericardial electrode 41 may be embedded between the flexible outer layer 20 and a patch of a secondary thin film 68, as demonstrated in FIG. 12. This patch may be made from the same material and may have approximately the same thickness as the thin film of the flexible outer layer. In other embodiments, the foil portion of electrode 41 may be embedded between two oval patches of thin film sized to be larger than the size of the foil electrode 41. Once assembled together, the combination of two patches of the thin film with the electrode 41 positioned in between thereof may, in turn, be fused or otherwise securely and sealingly attached to the flexible outer layer 20 in a secondary operation, as seen in FIG. 12.

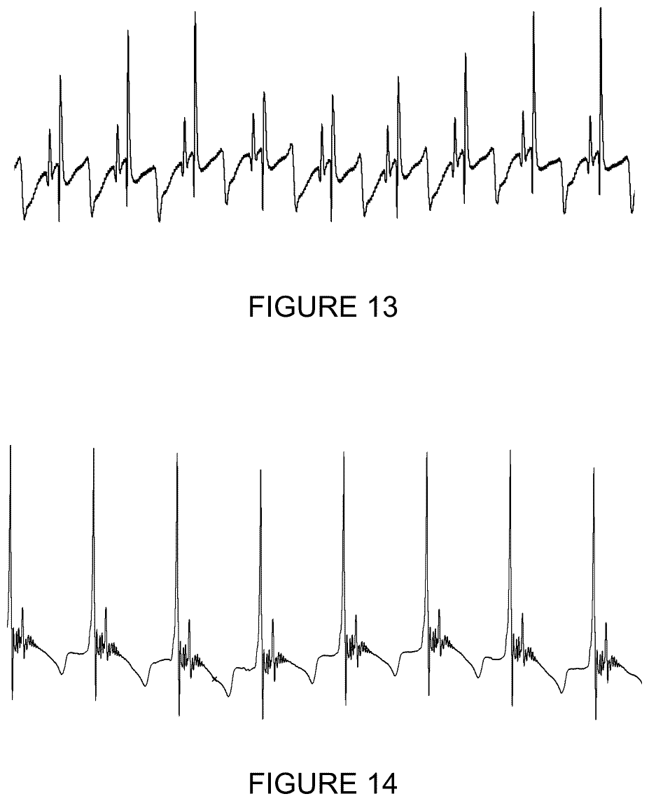

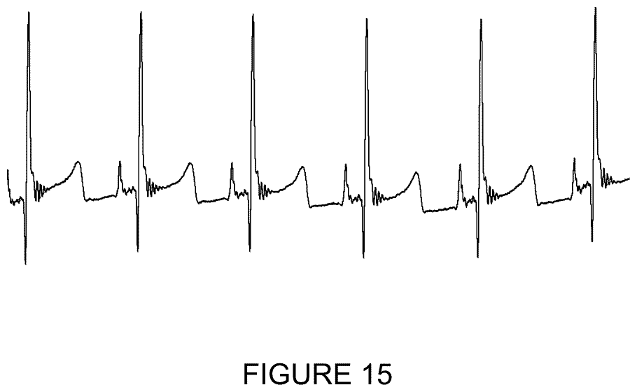

Finally, FIGS. 13, 14, and 15 illustrate typical examples of ECG recordings obtained from the skin, epicardial surface, and pericardial surface respectively collected during experimental evaluations of the devices of the present invention. As can be seen, all of these exemplary recordings are quite suitable for the purpose of providing timing information. This timing information can be used by the controller of the device for the automated operation of the actuator, configured to cause periodic inflation and deflation of the direct cardiac compression device of the invention in synchrony with the natural rhythm of the heart.

It is contemplated that any embodiment discussed in this specification can be implemented with respect to any method of the invention, and vice versa. It will be also understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference. Incorporation by reference is limited such that no subject matter is incorporated that is contrary to the explicit disclosure herein, no claims included in the documents are incorporated by reference herein, and any definitions provided in the documents are not incorporated by reference herein unless expressly included herein.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12, 15, 20 or 25%.

All of the devices and/or methods disclosed and claimed herein can be made and executed without undue experimentation in light of the present disclosure. While the devices and methods of this invention have been described in terms of preferred embodiments, it will be apparent to those of skill in the art that variations may be applied to the devices and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A direct cardiac compression device comprising:
a flexible outer layer configured to fit about at least a portion of a living heart,
wherein the flexible outer layer comprises a first pericardial electrode facing outward therefrom, the first pericardial electrode is configured to detect an electrocardiogram of the living heart,
at least one inflatable active chamber inside the flexible outer layer and facing the living heart,
a port operably connected with the at least one inflatable active chamber and configured to facilitate inflation and deflation of the at least one inflatable active chamber by an actuator to cause periodic direct compression of the living heart,
wherein the actuator is controlled to inflate and deflate the at least one inflatable active chamber using the electrocardiogram signal collected from the first pericardial electrode.

2. The direct cardiac compression device, as in claim 1, wherein the flexible layer is configured to fit about an apex of the living heart and at least a portion of a left ventricle and at least a portion of a right ventricle of the living heart.

3. The direct cardiac compression device, as in claim 1, further comprising a ground electrode.

4. The direct cardiac compression device, as in claim 3, wherein the outer layer extends from a hub containing the port and located adjacent the apex of the living heart, wherein the ground electrode is located on or adjacent to the hub.

5. The direct cardiac compression device, as in claim 1, wherein the first pericardial electrode is located at a midportion of the flexible outer layer, or at a basal portion of the flexible outer layer away from the apex of the living heart.

6. The direct cardiac compression device, as in claim 1, wherein the flexible outer layer comprises a second pericardial electrode facing outward therefrom, the second pericardial electrode is also configured to detect the electrocardiogram of the living heart, the second pericardial electrode is spaced apart from the first pericardial electrode.

7. The direct cardiac compression device, as in claim 6, wherein the flexible outer layer comprises a third pericardial electrode and a fourth pericardial electrode, wherein all pericardial electrodes are configured to face outward from the flexible outer layer and to detect the electrocardiogram of the living heart.

8. The direct cardiac compression device, as in claim 7, wherein the first, the second, the third, and the fourth pericardial electrodes are radially evenly spaced apart from each other along the flexible outer layer and are located respectively in a first quadrant, a second quadrant, a third quadrant, and a fourth quadrant thereof, thereby forming a plurality of redundant pericardial electrodes for collecting the electrogram of the living heart from a plurality of locations around the flexible outer layer.

9. The direct cardiac compression device, as in claim 1, wherein the first pericardial electrode comprises a first conductive foil securely embedded and sealed into the flexible outer layer, with at least a portion thereof exposed to and facing towards the pericardium for sensing the electrocardiogram.

10. The direct cardiac compression device, as in claim 9, wherein the first conductive foil is made from or coated with copper, gold, platinum, silver, steel, or alloys thereof.

11. The direct cardiac compression device, as in claim 9, wherein the first conductive foil has a thickness from 0.005 to 0.5 millimeters, thereby allowing the flexible outer layer to be folded prior to implantation of the direct cardiac compression device without damaging thereof.

12. The direct cardiac compression device, as in claim 9, wherein the first electrode further comprises a first electrode conductor attached to the first conductive foil and configured to transmit the electrocardiogram signal from the first pericardial electrode.

13. The direct cardiac compression device, as in claim 9, wherein the first electrode conductor is a multi-strand wire.

14. The direct cardiac compression device, as in claim 12, wherein the first electrode conductor is routed toward the hub inside the flexible outer layer.

15. The direct cardiac compression device, as in claim 14, wherein the first electrode conductor is attached to the flexible outer layer inside thereof in at least one location between the first pericardial electrode and the hub.

16. The direct cardiac compression device, as in claim 9, wherein the first pericardial electrode has a length greater than a width, with the length thereof oriented in a direction perpendicular to a radial circumference of the flexible outer layer.

17. The direct cardiac compression device, as in claim 16, wherein the first conductive foil has a general shape of an oval.

18. The direct cardiac compression device, as in claim 17, wherein the first conductive foil has a plurality of radial protrusions on a periphery thereof configured to facilitate retention of the first conductive foil within the flexible outer layer in a sealed manner.

19. A direct cardiac compression device configured to fit about at least a portion of a living heart, the direct cardiac compression device comprising:
one or more passive chambers facing the living heart and shaped to taper toward an apex of the living heart,
a plurality of inflatable active chambers externally adjacent to the one or more passive chambers, wherein inflatable active chambers are shaped to taper toward the apex of the living heart and at least partially surround the one or more passive chambers, wherein when inflated, the plurality of inflatable active chambers are configured to cause direct cardiac compression of the living heart,
a self-expandable wireframe positioned to surround and configured to support the plurality of inflatable active chambers, wherein the self-expandable wireframe extends from a hub,
a first port operably connected to the plurality of inflatable active chambers to independently inflate and deflate thereof,
a second port operably connected to the at least one or more passive chambers to inject or adjust a volume of fluid therein, and
a flexible outer layer configured to surround the one or more passive chambers, the plurality of active chambers, and the self-expandable frame,
wherein the outer layer comprises a first pericardial electrode facing outward therefrom, the first pericardial electrode is configured to detect an electrocardiogram of the living heart.

20. A direct cardiac compression device comprising:
a flexible outer layer comprises an outer surface and an inner surface, wherein the inner surface is configured to fit about at least a portion of a living heart; a first pericardial electrode in contact with the outer surface and facing away from the inner surface, wherein the first pericardial electrode is configured to detect an electrocardiogram of the living heart; at least one inflatable active chamber inside the flexible outer layer and facing the living heart, a port operably connected with the at least one inflatable active chamber and configured to facilitate inflation and deflation of the at least one inflatable active chamber by an actuator to cause periodic direct compression of the living heart, wherein the actuator is controlled to inflate and deflate the at least one active chamber using the electrocardiogram signal collected from the first pericardial electrode.
